(19) United States
(12) Patent Application Publication
(10) Pub. No.: US 2026/0250221 A1
(43) Pub. Date: Aug. 27, 2026

(54) METHOD FOR PURIFYING 1,1,1,2,3,3-HEXAFLUOROPROPANE

(71) Applicant: ARKEMA FRANCE, Colombes (FR)

(72) Inventors: Kevin HISLER, Pierre-Benite (FR); Jérémy RETALI, Pierre-Benite (FR); Pierre-Marie SEDAT, Pierre-Benite (FR); Géraldine CAVALLINI, Pierre-Benite (FR)

(73) Assignee: ARKEMA FRANCE, Colombes (FR)

(21) Appl. No.: 18/993,023

(22) PCT Filed: May 7, 2023
(86) PCT No.: PCT/FR2023/051033

§ 371 Date: Jan. 10, 2025

(30) Foreign Application Priority Data
Jul. 12, 2022 (FR) FR2207139

(51) Int. Cl. C07C 17/38; C07C 17/354

(52) U.S. Cl. CPC C07C 17/38; C07C 17/354

(57) ABSTRACT

The present invention relates to a process for purifying 1,1,1,2,3,3-hexafluoropropane from a mixture comprising 1,1,1,2,3,3-hexafluoropropane and at least one contaminant selected from the group consisting of hydrogen, nitrogen and oxygen, said process 5 comprising a step (a) of bringing said mixture into contact with a membrane in order to form a flow comprising said 1,1,1,2,3,3-hexafluoropropane and a flow comprising said at least one contaminant.

### TECHNICAL FIELD

The present invention relates to a process for purifying hydrofluoroalkanes. Preferably, the present invention relates to a process for purifying hydrofluoroalkanes by membrane separation.

### TECHNOLOGICAL BACKGROUND OF THE INVENTION

1,1,1,2,3,3-Hexafluoropropane (HFC-236ea) is a hydrofluorocarbon and has been described as a starting material for the manufacture of 1,1,1,2,3-pentafluoropropene or as an intermediate in the manufacture of 1,1,1,2,3-pentafluoropropane and/or of 2,3,3,3-tetrafluoropropene. Mention may be made in particular of documents U.S. Pat. Nos. 5,679,875, 539,600, 8,359,964 and 8,389,779.

It can be prepared by high-temperature pyrolysis of chlorodifluoromethane (CHCIF2) in the presence of 1,1,1,2-tetrafluoroethane. Mention may be made, for example, of the document WO 1996029296.

1,1,1,2,3,3-Hexafluoropropane can also be prepared by a process during which at least one tetrafluorochloropropene is obtained from the dechlorofluorination of 1,1,1,2,2-pentafluoro-3,3-dichloropropane (HCFC-225ca) and/or 1,1,2,2,3-pentafluoro-1,3-dichloropropane (HCFC-225cb) with hydrogen in the presence of a catalyst consisting of a metal oxide. Then, the tetrafluorochloropropene(s) produced (1,1,1,2-tetrafluoro-3-chloro-2-propene (HCFO-1224 yd), 1,1,2,3-tetrafluoro-1-chloro-2-propene (HCFO-1224ye) and 1,1,2,3-tetrafluoro-3-chloro-1-propene (HCFO-1224yc)) is or are subsequently fluorinated in the presence of a catalyst to give HFC-236ea. Mention may be made for example of document U.S. Pat. No. 5,532,418.

Lastly, according to U.S. Pat. No. 5,563,304, 1,1,1,2,3,3-hexafluoropropane can be prepared by reaction of 1,2,3,3,3-pentafluoropropene (HFO-1225ye) with hydrogen fluoride at a high temperature, on a catalyst chosen from the group comprising aluminum fluoride, fluorinated aluminum oxide, aluminum fluoride-supported metals, fluorinated aluminum oxide-supported metals and catalysts comprising trivalent chromium.

In particular, 1,1,1,2,3,3-hexafluoropropane can be prepared by catalytic hydrogenation of hexafluoropropene. This reaction is generally carried out with an excess of hydrogen. The hydrogen is generally removed by several distillation steps and drying steps prior to the distillation.

1,1,1,2,3,3-Hexafluoropropane can be used as a cleaning agent in the semiconductor industry. In this type of application, the 1,1,1,2,3,3-hexafluoropropane must be of high purity. There is therefore a need to provide a process for purifying 1,1,1,2,3,3-hexafluoropropane that makes it possible to remove certain contaminants resulting from the processes for the preparation thereof.

### SUMMARY OF THE INVENTION

According to a first aspect, the present invention relates to a process for purifying 1,1,1,2,3,3-hexafluoropropane from a mixture comprising 1,1,1,2,3,3-hexafluoropropane and hydrogen, said process comprising a step (a) of bringing said mixture into contact with a membrane M1 in order to form a flow F1 comprising said 1,1,1,2,3,3-hexafluoropropane and a flow F2 comprising hydrogen, characterized in that said membrane M1 is made of a material selected from the group consisting of polyolefin, polyether, polyimide, polyvinylidene fluoride, a cellulose-based material and poly(methyl methacrylate).

According to a preferred embodiment, said membrane M1 is made of a material selected from the group consisting of polyolefin and polyether.

According to a preferred embodiment, said membrane M1 is made of a material selected from the group consisting of polyethylene, polypropylene, polymethylpentene, poly[oxy(2,6-dimethyl-1,4-phenylene)] and poly(phenylene oxide).

According to a preferred embodiment, said membrane M1 has a selectivity of greater than 100, said selectivity being calculated by the ratio between the permeability of hydrogen and the permeability of said 1,1,1,2,3,3-hexafluoropropane through said membrane M1; the selectivity is preferably greater than 250, and in particular greater than 500.

According to a preferred embodiment, said mixture contains less than 100 ppm of water based on the total weight of said mixture.

According to a preferred embodiment, the content by mass of hydrogen in said mixture is less than 25% by weight based on the total weight of said mixture.

According to a preferred embodiment, said mixture is obtained from a reaction of hydrogenation of hexafluoropropene, which has optionally been purified beforehand, preferably by distillation.

According to another aspect, the present invention relates to a process for producing 1,1,1,2,3,3-hexafluoropropane, comprising the steps of:
A) gas-phase hydrogenation of hexafluoropropene in the presence of hydrogen and of a hydrogenation catalyst in order to form a stream A1 comprising 1,1,1,2,3,3-hexafluoropropane and unreacted hydrogen;
B) optionally purification of the stream A1 in order to form a purified stream A2;
C) implementation of the purification process according to the present invention using said stream A1 or said stream A2.

According to another aspect, the present invention relates to a process for purifying 1,1,1,2,3,3-hexafluoropropane from a mixture comprising 1,1,1,2,3,3-hexafluoropropane and at least one contaminant selected from the group consisting of nitrogen and oxygen or a mixture thereof, said process comprising a step (a) of bringing said mixture into contact with a membrane M1′ in order to form a flow F1′ comprising said 1,1,1,2,3,3-hexafluoropropane and a flow F2′ comprising said at least one contaminant.

According to a preferred embodiment, said membrane M1′ is made of a material selected from the group consisting of polyolefin, polyether, polyimide, polyaramid, polyamide, polysulfone, polyvinylidene fluoride, poly(methyl methacrylate), polytetrafluoroethylene, polyvinyl fluoride, polychlorotrifluoroethylene, polyethylene-tetrafluoroethylene or tetrafluoroethylene/perfluorovinyl ether copolymer optionally substituted by an SO₃H group, of a cellulose-based material and of a material containing a siloxane functional group.

According to a preferred embodiment, said contaminant is oxygen and said membrane M1′ is made of a material selected from the group consisting of polyolefin, polyether, polyimide, polyvinylidene fluoride, a cellulose-based material, polyalkylsiloxane and poly(methyl methacrylate).

According to a preferred embodiment, said membrane M1′ is made of a material selected from the group consisting of polyethylene, polypropylene, polymethylpentene, poly[oxy(2,6-dimethyl-1,4-phenylene)], poly(phenylene oxide), cellulose acetate, polyimide and polydimethylsiloxane.

According to a preferred embodiment, said contaminant is nitrogen and said membrane M1′ is made of a material selected from the group consisting of polyolefin and of polyether, preferably polyethylene, polypropylene, polymethylpentene, poly[oxy(2,6-dimethyl-1,4-phenylene)] or poly(phenylene oxide), in particular polypropylene, poly(phenylene oxide) or polymethylpentene.

According to a preferred embodiment, the content by mass of said contaminant in said mixture is less than 5% based on the total weight of said mixture, preferably less than 1% based on the total weight of said mixture.

According to another aspect, the present invention relates to a process for purifying 1,1,1,2,3,3-hexafluoropropane from a mixture comprising 1,1,1,2,3,3-hexafluoropropane, nitrogen, hydrogen and optionally oxygen, said process comprising a step (a) of bringing said mixture into contact with a membrane M2 in order to form a flow F3 comprising said 1,1,1,2,3,3-hexafluoropropane and a flow F4 comprising nitrogen, hydrogen and optionally oxygen, said membrane M2 being made of a material selected from the group consisting of polyethylene, polypropylene, polymethylpentene, poly[oxy(2,6-dimethyl-1,4-phenylene)] and poly(phenylene oxide).

### DETAILED DESCRIPTION OF THE INVENTION

### Separation of 1,1,1,2,3,3-Hexafluoropropane from Hydrogen

According to a first aspect, the present invention relates to a process for purifying 1,1,1,2,3,3-hexafluoropropane from a mixture comprising 1,1,1,2,3,3-hexafluoropropane and hydrogen. Said process comprises a step (a) of bringing said mixture into contact with a membrane M1 in order to form a flow F1 comprising said 1,1,1,2,3,3-hexafluoropropane and a flow F2 comprising hydrogen.

Preferably, said mixture comprises a molar content of hydrogen of less than 50%, preferably of less than 25%, in particular of less than 15%, based on the total molar amount of the mixture. Preferably, said mixture comprises a molar content of hydrogen of greater than 0.5%, preferably of greater than 1%, based on the total molar amount of the mixture.

Preferably, said mixture is in gaseous form.

The present process thus makes it possible to produce a flow F1 enriched in 1,1,1,2,3,3-hexafluoropropane with respect to the initial mixture before being brought into contact with the membrane. Preferably, said flow F1 has a reduced molar content of hydrogen with respect to said mixture.

According to a preferred embodiment, said flow F1 comprises at least 25% by weight of 1,1,1,2,3,3-hexafluoropropane, advantageously at least 30% by weight of 1,1,1,2,3,3-hexafluoropropane, preferably at least 35% by weight of 1,1,1,2,3,3-hexafluoropropane, more preferentially at least 40% by weight of 1,1,1,2,3,3-hexafluoropropane, in particular at least 45% by weight of 1,1,1,2,3,3-hexafluoropropane, more particularly at least 50% by weight of 1,1,1,2,3,3-hexafluoropropane, based on the total weight of said flow F1.

Preferably, said flow F1 comprises less than 20% by weight of hydrogen, based on the total weight of said flow F1. Advantageously, said flow F1 comprises less than 15% by weight of hydrogen, preferably less than 10% by weight, in particular less than 5% by weight, more particularly less than 1% by weight of hydrogen, based on the total weight of said flow F1.

In the present process, the flow F2 is enriched in hydrogen. According to a preferred embodiment, said flow F2 has an increased molar content of hydrogen with respect to said mixture. Preferably, said flow F2 comprises at least 25% by weight of hydrogen, more preferentially at least 50% by weight of hydrogen, in particular at least 75% by weight of hydrogen, more particularly at least 80% by weight of hydrogen, favorably at least 95% by weight of hydrogen, based on the total weight of said flow F2.

In the present patent application, the term membrane refers to a membrane which is selectively permeable to one or more compounds so that it makes it possible for the different compounds to migrate through the membrane at different flow rates. The membrane restricts the movement of the molecules which pass through it so that some molecules move more slowly than others or are completely excluded (that is to say, impermeable). For example, the membrane can be selectively permeable to hydrogen and impermeable (or weakly permeable) to 1,1,1,2,3,3-hexafluoropropane.

The permeability of a membrane depends on its ability to limit or not limit the diffusion of these compounds through the membrane. The membranes can selectively separate the components over a wide range of solubility parameters and molecular sizes, from macromolecular materials to simple ionic or covalent compounds. The determining property for the performance qualities of the membrane is mainly the selectivity. The membrane separation process is characterized by the fact that a feed flow is divided into two flows: retentate and permeate. The retentate is the portion of the feed which does not pass (or only slightly passes) through the membrane, while the permeate is the portion of the feedstock which passes through the membrane.

In the present patent application, the retentate may be one of the flows described as a function of the membrane used and of the compounds under consideration.

Unlike distillation processes, membrane separation does not require phase separation, which generally makes possible significant savings in energy, compared with distillation processes. The capital costs can also be reduced because membrane separation processes generally have no moving parts, no complex control schemes and few items of auxiliary equipment, compared with other separation processes known in the art.

The membranes can be produced with an extremely high selectivity for the components to be separated. In general, the values of the selectivity are much higher than the typical values of relative volatility for distillation operations. Membrane separation processes may also be able to recover minor but valuable components from the main flow without substantial energy cost. Membrane separation processes are potentially better for the environment since the membrane approach requires the use of relatively simple and nonharmful materials.

According to a preferred embodiment, said membrane M1 is made of a material selected from the group consisting of polyolefin, polyether, polyimide, polyvinylidene fluoride, a cellulose-based material and poly(methyl methacrylate). Preferably, said membrane M1 is made of a material selected from the group consisting of polyolefin and polyether. In particular, said membrane M1 is made of a material selected from the group consisting of polyethylene, polypropylene, polymethylpentene, poly[oxy(2,6-dimethyl-1,4-phenylene)] and poly(phenylene oxide).

In the present patent application, the term polyether refers in particular to a polyaryl ether comprising the monomeric unit —[—O—Ar—]— or —[—Ar¹—O—Ar²—]— in which Ar, Ar¹ and Ar² are, independently of one another, an aromatic ring comprising from 6 to 12 carbon atoms optionally substituted by one or more C1-C10 alkyl functional groups; preferably, Ar is a phenyl group optionally substituted by one, two, three or four C1-C3 alkyl functional groups. In particular, the polyether is poly[oxy(2,6-dimethyl-1,4-phenylene)] or poly(phenylene oxide).

In the present patent application, the term cellulose refers to a polymer consisting of a linear chain of D-glucose units the hydroxyl function of which is optionally, partially or not partially, substituted. The hydrogen of one or more hydroxyl groups may be substituted by a —C(O)—R or C1-C5 alkyl group optionally substituted by an OH, CO2H, CO2R group, with R═C1-C5 alkyl. Preferably, the cellulose is preferably cellulose acetate.

In the present patent application, the term polyolefin refers in particular to polyethylene, polypropylene, polymethylpropene, polybutene, polypentene, polymethylpentene, polymethylbutene, polyhexene, polymethylpentene and polyethylbutene.

In general, it can be considered that there is separation between the hydrogen and the 1,1,1,2,3,3-hexafluoropropane when the selectivity is greater than 2. The higher the selectivity, the more efficient the separation. The process is particularly efficient when the selectivity is greater than or equal to 5, preferably greater than or equal to 10, in particular greater than or equal to 20.

When the permeability of said membrane M1 with regard to the hydrogen is greater than the permeability of said membrane M1 with regard to the 1,1,1,2,3,3-hexafluoropropane, the selectivity is calculated by the ratio of the permeability of the hydrogen to the permeability of said 1,1,1,2,3,3-hexafluoropropane under consideration through said membrane M1, i.e. selectivity=[permeability of the hydrogen]/[permeability of the 1,1,1,2,3,3-hexafluoropropane].

Preferably, said membrane M1 has a selectivity of greater than 4, advantageously of greater than 5, preferably of greater than 6, more preferentially of greater than 7, in particular of greater than 8, more particularly of greater than 9, said selectivity being calculated by the ratio of the permeability of the hydrogen to the permeability of 1,1,1,2,3,3-hexafluoropropane through the membrane. In particular, the selectivity of said membrane M1 may be greater than 10, or greater than 15, or greater than 20, or greater than 25, or greater than 30, or greater than 35, or greater than 40, or greater than 45, or greater than 50, or greater than 55, or greater than 60, or greater than 65, or greater than 70, or greater than 75, or greater than 80, or greater than 85 or greater than 90 or greater than 95 or greater than 100 or greater than 105 or greater than 110 or greater than 115 or greater than 120 or greater than 125 or greater than 130 or greater than 135 or greater than 140 or greater than 145 or greater than 150, said selectivity being calculated by the ratio of the permeability of the hydrogen to the permeability of said 1,1,1,2,3,3-hexafluoropropane through said membrane M1, preferably when said membrane is made of polyolefin or polyether.

According to a particular embodiment, said membrane M1 has a selectivity of greater than 200, advantageously of greater than 250, preferably of greater than 300, more preferentially of greater than 400, in particular of greater than 500, said selectivity being calculated by the ratio of the permeability of the hydrogen to the permeability of said 1,1,1,2,3,3-hexafluoropropane through said membrane M1, preferably when said membrane is made of polymethylpentene, poly[oxy(2,6-dimethyl-1,4-phenylene)] and poly(phenylene oxide).

According to a preferred embodiment, in the present process, the mixture of step (a) has a content by mass of water of less than 1000 ppm, advantageously 500 ppm, preferably of less than 200 ppm, more preferentially of less than 100 ppm, in particular of less than 50 ppm, more particularly of less than 10 ppm, based on the total weight of the mixture.

According to a preferred embodiment, said mixture of step a) is obtained from a reaction of hydrogenation of hexafluoropropene, which has optionally been purified beforehand, preferably by distillation. This is detailed in particular below in the section concerning the production of the 1,1,1,2,3,3-hexafluoropropane.

According to a preferred embodiment, said membrane M1 is chosen from a film, a laminated structure, hollow fibers and coated fibers.

Step (a) may be carried out over a wide temperature and pressure range.

Preferably, step (a) of bringing said mixture into contact with said membrane M1 is carried out at a pressure of from 0.1 bara to 30 bara, advantageously from 0.2 bara to 25 bara, preferably from 0.3 bara to 20 bara, more preferentially from 0.4 bara to 15 bara, in particular from 0.5 bara to 10 bara, more particularly from 0.5 bara to 5 bara.

Preferably, step (a) of bringing said mixture into contact with said membrane M1 is carried out at a temperature of from 0° C. to 150° C., advantageously from 0° C. to 125° C., preferably from 5° C. to 100° C., more preferentially from 10° C. to 75° C., in particular from 10° C. to 50° C.

During the implementation of the process, a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa.

### Separation of 1,1,1,2,3,3-Hexafluoropropane from Nitrogen and/or Oxygen

According to another aspect of the present invention, a process for purifying 1,1,1,2,3,3-hexafluoropropane from a mixture comprising 1,1,1,2,3,3-hexafluoropropane and at least one contaminant selected from the group consisting of nitrogen and oxygen or a mixture thereof is provided.

Preferably, said process comprises a step (a) of bringing said mixture into contact with a membrane M1′ in order to form a flow F1′ comprising said 1,1,1,2,3,3-hexafluoropropane and a flow F2′ comprising said at least one contaminant.

In general, said membrane M1′ may made of a material selected from the group consisting of polyolefin, polyether, polyimide, polyaramid, polyamide, polysulfone, polyvinylidene fluoride, poly(methyl methacrylate), polytetrafluoroethylene, polyvinyl fluoride, polychlorotrifluoroethylene, polyethylene-tetrafluoroethylene or tetrafluoroethylene/perfluorovinyl ether copolymer optionally substituted by an SO₃H group, of a cellulose-based material and of a material containing a siloxane functional group.

According to another particular embodiment, said at least one contaminant is nitrogen. In this case, said membrane M1′ is preferably made of a material selected from the group consisting of polyolefin and of polyether. The terms polyolefin and polyether are as defined above. Preferably, said membrane M1′ is made of a material selected from the group consisting of polyethylene, polypropylene, polymethylpentene, poly[oxy(2,6-dimethyl-1,4-phenylene)] or poly(phenylene oxide). In particular, said membrane M1′ is made of a material selected from the group consisting of polypropylene or polymethylpentene. Said membrane M1′ has, preferably, a selectivity of greater than 5, said selectivity being calculated by the ratio of the permeability of the nitrogen to the permeability of said 1,1,1,2,3,3-hexafluoropropane through said membrane M1′. Advantageously, said membrane M1′ has a selectivity of greater than 10, preferably of greater than 20, more preferentially of greater than 50, in particular of greater than 75, said selectivity being calculated by the ratio of the permeability of the nitrogen to the permeability of said 1,1,1,2,3,3-hexafluoropropane through said membrane M1′.

According to another particular embodiment, said at least one contaminant is oxygen. In this case, said membrane M1′ has, preferably, a selectivity of greater than 5, said selectivity being calculated by the ratio of the permeability of the oxygen to the permeability of said 1,1,1,2,3,3-hexafluoropropane through said membrane M1′. Advantageously, said membrane M1′ has a selectivity of greater than 10, preferably of greater than 20, more preferentially of greater than 50, in particular of greater than 75, said selectivity being calculated by the ratio of the permeability of the oxygen to the permeability of said 1,1,1,2,3,3-hexafluoropropane through said membrane M1′. Preferably, said membrane M1′ is made of a material selected from the group consisting of polyolefin, polyether, polyimide, polyvinylidene fluoride, a cellulose-based material, polyalkylsiloxane and poly(methyl methacrylate). In particular, said membrane M1′ is made of a material selected from the group consisting of polypropylene, polymethylpentene, poly[oxy(2,6-dimethyl-1,4-phenylene)] and poly(phenylene oxide).

Step (a) may be carried out over a wide temperature and pressure range.

Preferably, step (a) of bringing said mixture into contact with said membrane M1′ is carried out at a pressure of from 0.1 bara to 30 bara, advantageously from 0.2 bara to 25 bara, preferably from 0.3 bara to 20 bara, more preferentially from 0.4 bara to 15 bara, in particular from 0.5 bara to 10 bara, more particularly from 0.5 bara to 5 bara.

Preferably, step (a) of bringing said mixture into contact with said membrane M1′ is carried out at a temperature of from 0° C. to 150° C., advantageously from 0° C. to 125° C., preferably from 5° C. to 100° C., more preferentially from 10° C. to 75° C., in particular from 10° C. to 50° C.

During the implementation of the process, a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa.

According to a preferred embodiment, in the present process, the mixture of step (a) is in anhydrous form. The term anhydrous refers to a content by mass of water of less than 1000 ppm, advantageously 500 ppm, preferably of less than 200 ppm, in particular of less than 100 ppm, based on the total weight of the mixture.

According to a preferred embodiment, said membrane M1′ is chosen from a film, a laminated structure, hollow fibers and coated fibers.

According to a particular embodiment, the content by mass of said contaminant in said mixture subjected to step (a) is less than 15% based on the total weight of said mixture, advantageously less than 10%, preferably less than 5%, in particular less than 1%, based on the total weight of said mixture. According to a particular embodiment, the content by mass of 1,1,1,2,3,3-hexafluoropropane in said mixture subjected to step (a) is greater than 50% based on the total weight of said mixture, advantageously greater than 70%, preferably greater than 80%, in particular greater than 90%, based on the total weight of said mixture. Preferably, said mixture is in gaseous form.

The present process thus makes it possible to produce a flow F1′ enriched in 1,1,1,2,3,3-hexafluoropropane with respect to the initial mixture before being brought into contact with the membrane M1′. Preferably, said flow F1′ has a reduced molar content of said contaminant with respect to said mixture. According to a preferred embodiment, said flow F1′ comprises at least 60% by weight of 1,1,1,2,3,3-hexafluoropropane, advantageously at least 70% by weight of 1,1,1,2,3,3-hexafluoropropane, preferably at least 80% by weight of 1,1,1,2,3,3-hexafluoropropane, more preferentially at least 90% by weight of 1,1,1,2,3,3-hexafluoropropane, in particular at least 95% by weight of 1,1,1,2,3,3-hexafluoropropane, based on the total weight of said flow F1′. Preferably, said flow F1′ comprises less than 10% by weight of said contaminant based on the total weight of said flow F1′. Advantageously, said flow F1′ comprises less than 5% by weight of said contaminant, preferably less than 1% by weight, in particular less than 0.5% by weight, more particularly less than 0.1% by weight of said contaminant, based on the total weight of said flow F1′. In the present process, the flow F2′ is enriched in said contaminant. According to a preferred embodiment, said flow F2′ has an increased molar content of hydrogen with respect to said mixture. Preferably, said flow F2′ comprises at least 25% by weight of said contaminant, more preferentially at least 50% by weight of said contaminant, in particular at least 75% by weight of said contaminant, more particularly at least 80% by weight of said contaminant, favorably at least 95% by weight of said contaminant, based on the total weight of said flow F2′.

### Separation of 1,1,1,2,3,3-Hexafluoropropane from a Mixture Comprising Nitrogen, Hydrogen and Optionally Oxygen

According to another aspect, the present invention provides a process for purifying 1,1,1,2,3,3-hexafluoropropane from a mixture comprising 1,1,1,2,3,3-hexafluoropropane, nitrogen, hydrogen and optionally oxygen. Preferably, said process comprises a step (a) of bringing said mixture into contact with a membrane M2 in order to form a flow F3 comprising said 1,1,1,2,3,3-hexafluoropropane and a flow F4 comprising nitrogen, hydrogen and optionally oxygen. In particular, said membrane M2 is made of a material selected from the group consisting of polyolefin and polyether. According to a preferred embodiment, said membrane M2 is made of a material selected from the group consisting of polyethylene, polypropylene, polymethylpentene, poly[oxy(2,6-dimethyl-1,4-phenylene)] and poly(phenylene oxide).

Step (a) may be carried out over a wide temperature and pressure range.

Preferably, step (a) of bringing said mixture into contact with said membrane M2 is carried out at a pressure of from 0.1 bara to 30 bara, advantageously from 0.2 bara to 25 bara, preferably from 0.3 bara to 20 bara, more preferentially from 0.4 bara to 15 bara, in particular from 0.5 bara to 10 bara, more particularly from 0.5 bara to 5 bara.

Preferably, step (a) of bringing said mixture into contact with said membrane M2 is carried out at a temperature of from 0° C. to 150° C., advantageously from 0° C. to 125° C., preferably from 5° C. to 100° C., more preferentially from 10° C. to 75° C., in particular from 10° C. to 50° C.

During the implementation of the process, a pressure difference is observed between the inlet of the membrane and the outlet of the membrane. The differential pressure expressed here corresponds to the pressure difference existing between the inlet and the outlet of said membrane. Preferably, the differential pressure is from 1 to 3000 kPa, preferably from 50 to 2000 kPa, in particular from 100 to 1000 kPa, more particularly from 100 to 500 kPa.

According to a preferred embodiment, in the present process, the mixture of step (a) is in anhydrous form. The term anhydrous refers to a content by mass of water of less than 1000 ppm, advantageously 500 ppm, preferably of less than 200 ppm, in particular of less than 100 ppm, based on the total weight of the mixture.

According to a preferred embodiment, said membrane M2 is chosen from a film, a laminated structure, hollow fibers and coated fibers.

Said membrane M2 has, preferably, a selectivity of greater than 5, advantageously of greater than 10, preferably of greater than 20, more preferentially of greater than 50, in particular of greater than 75, said selectivity being calculated by the ratio of the permeability of the nitrogen or of the hydrogen or of the oxygen to the permeability of said 1,1,1,2,3,3-hexafluoropropane through said membrane M2, as is demonstrated above.

### Process for Producing and Purifying 1,1,1,2,3,3-Hexafluoropropane

According to another aspect, the present invention provides a process for producing 1,1,1,2,3,3-hexafluoropropane. Said process comprises the steps of:
A) gas-phase hydrogenation of hexafluoropropene in the presence of hydrogen and of a hydrogenation catalyst in order to form a stream A1 comprising 1,1,1,2,3,3-hexafluoropropane and unreacted hydrogen;
B) optionally purification of the stream A1 in order to form a purified stream A2;
C) implementation of the purification process according to the present invention using said stream A1 or said stream A2.

Preferably, the hydrogenation step A) is carried out in the presence of a catalyst. As catalyst, mention may especially be made of metals such as Pd, Ru, Pt, Rh, Ir, Fe, Co, Ni, Cu, Ag, Re, Os, Au, Ge, Te, optionally supported. As support, mention may especially be made of carbon, alumina, fluorinated alumina, AlF₃, oxides, oxyfluorides and fluorides of Cr, Ti, Zr, Mg and Zn, silica and silicon carbide. The amount of metals present in the catalyst, when the latter is supported, may be between 0.001% and 10% by weight, preferably between 0.001% and 1.0% by weight, in particular from 0.01% to 0.2% by weight. The hydrogenation step is advantageously carried out in the presence of Pd supported on alumina, preferably in the alpha polymorphic form. In particular, said hydrogenation catalyst may comprise Pd supported on alumina in the alpha polymorphic form, the palladium representing between 0.001% and 1.0% by weight, preferably from 0.01% to 0.2% based on the total weight of the catalyst. The hydrogenation step may be carried out either in liquid phase or in gas phase. Gas phase is, however, preferred.

The hydrogenation step A) is carried out in the presence of hydrogen, advantageously with a hydrogen/hexafluoropropene molar ratio of between 1 and 50 and most particularly between 2 and 15.

The hydrogenation step A) is preferably carried out at a temperature of between 50 and 200° C., preferably of between 8° and 120° C. Preferably, the temperature at the inlet of the reactor for the hydrogenation step A) is between 3° and 100° C., advantageously between 4° and 80° C.

The contact time for the hydrogenation step A), defined as the ratio of the volume of the catalyst bed to the volume flow rate of the total flow under standard temperature and pressure conditions, is preferably between 0.1 s and 20 s, and advantageously between 0.5 s and 5 s.

The hydrogenation step A) is preferably carried out at an absolute pressure of between 0.5 and 20 bar and advantageously of between 1 and 5 bar.

Preferably, the hydrogenation step A) is carried out in the presence of a diluent which may be introduced along with the reactants into the reaction medium. The diluent is an inert gas which does not react under the conditions of the hydrogenation step. As diluent, mention may be made of nitrogen, helium or argon. The molar ratio of diluent/reactants at the inlet of the reactor for the hydrogenation step A) may be between 100:1 and 1:1, preferably between 75:1 and 1:1, advantageously between 50:1 and 1:1.

In particular, during step A) according to the present invention, the diluent may be the hydrogenation product which is HFC-236ea. In this case, one portion of the gaseous effluent obtained from the reactor, comprising HFC-236ea, unreacted hydrogen and possibly unreacted hexafluoropropene, is recycled and the other portion of the gaseous effluent obtained from the reactor is subjected to a separation and/or purification step. The gas flow comprising the recycle loop and the reactants may be preheated before introduction into the reactor. The portion of the gaseous effluent recycled into the reactor preferably represents at least 90% by volume of the total amount of effluent at the reactor outlet, advantageously at least 93% by volume. Particularly preferably, the portion of the effluent recycled into the reactor represents between 94% and 98% by volume of the total effluent at the reactor outlet.

Stream A1 may be purified before carrying out step C). The purification may be a drying step. The drying step may be carried out by bringing said stream into contact with a solid absorbing agent. Said solid absorbent may comprise an agent which absorbs acidic molecules and/or a water-absorbing agent. Said water-absorbing agent may be an inorganic salt such as magnesium sulfate, calcium sulfate, calcium chloride or may be a molecular sieve of 3 A, 4 A, 5 A, AW500, XH-7, XH-9 or 13X type, silica gel, activated carbon or a mixture thereof. Said agent which absorbs acidic molecules may be a metal oxide such as aluminum oxide, an alkaline-earth metal oxide, an alkali metal oxide or the hydroxide of a metal such as aluminum hydroxide, an alkaline-earth metal hydroxide, an alkali metal hydroxide, aluminosilicates such as andalusite, kyanite, sillimanite, calcium aluminosilicate, sodium aluminosilicate or silica or a mixture thereof. When the drying step is carried out in the presence of a water-absorbing agent and an agent which absorbs acidic molecules, said stream is preferentially brought into contact with the agent which absorbs acidic molecules and then with the water-absorbing agent. The agent which absorbs acidic molecules preferably absorbs hydrofluoric acid.

According to another embodiment, the purification comprises a step of condensing the stream A1. The stream A1 on conclusion of the hydrogenation step A) may be subjected to a condensation step under conditions such that the unreacted hydrogen is not condensed and that a portion of the HFC-236a formed in step A) is condensed. Preferably, the condensation step is carried out at a temperature of between 0 and 50° C. and at a pressure of between 0.5 and 20 bar absolute, advantageously between 1 and 5 bar absolute.

Preferably, the condensation step is carried out under conditions such that between 1% and 30% of the HFC-236ea leaving the reactor is condensed and advantageously between 2% and 10% is condensed. The uncondensed fraction may then be recycled into the hydrogenation step A) after optional heating. The uncondensed fraction may be subjected to step C) of the present process. The uncondensed fraction comprises 1,1,1,2,3,3-hexafluoropropane and hydrogen.

The condensed fraction, thus recovered and purified, may then be evaporated before being sent to step C). The condensed fraction may be said purified stream A2. Before carrying out step C), the condensed fraction may be purified and/or dried. The condensed fraction comprises 1,1,1,2,3,3-hexafluoropropane and hydrogen. However, the hydrogen is preferably present in small proportions.

According to another embodiment, step B) may be a distillation step for recovering a stream A2 comprising 1,1,1,2,3,3-hexafluoropropane and hydrogen, said stream A2 being enriched in 1,1,1,2,3,3-hexafluoropropane with respect to the stream A1.

Alternatively, a distillation step may be carried out after step C). In this case, the flow F1 as defined in the present application may be distilled.

### EXAMPLES

The permeability of a gaseous compound through a polymer is measured using an Evonik MET Crossflow Filtration Cell (with an internal diameter of 52 mm and an active surface area of 14 cm²) for the polymers in the form of a film or using a commercial module for the polymers in the form of fibers. The polyimide film is a Dupont Kapton HN film, the polymethylpentene film has the reference MX004, the silicone has the reference USP Class VI. The films are provided by Goodfellow.

In the examples below, the membranes tested are in the form of a film with an active surface area of 14 cm² and the thickness of which is shown in table 1 below.

TABLE 1

| Material | Thickness |
|---|---|
| Polyimide | 25 μm |
| Cellulose acetate | 35 μm |
| PMP | 50 μm |
| Polypropylene | 25 μm |
| PPO | 50 μm |

PMP = polymethylpentene;
PMMA: polymethyl methacrylate;
PPO = poly(phenylene oxide)

The permeability is calculated according to the following formula: P=Q×e×S⁻¹×ΔP⁻¹
with P: permeability in cm²·s⁻¹·Pa⁻¹
   Q: permeate flow rate in cm³/s
   e: thickness of the membrane in cm
   S: surface area of the membrane in cm²
   ΔP: pressure difference across the membrane in Pa (i.e. differential pressure mentioned in the present patent application)

The permeability is generally expressed in barrer (10⁻¹⁰·cm³(STP)·cm·cm²·s⁻¹·cmHg⁻¹) according to the conversion:

$${P}_{barrer} = P \times {10}^{10} / ( 7.500615 \times {10}^{- 4} )$$

Thus, it is possible to calculate the permeability of a compound through a material from the data of the material (surface area, thickness), the pressure difference across the membrane and the measurement of the permeate flow rate through the membrane. The permeability is thus measured by keeping under pressure a compound upstream of the membrane in the absence of an outlet on the retentate side, and by measuring the flow rate of this same compound at atmospheric pressure on the permeate side. The pressure difference corresponds to the difference between the pressure upstream of the membrane and the pressure downstream of the membrane, in this case atmospheric pressure. The tests are carried out at a temperature of 25° C., except for the silicone, the tests of which were carried out at 35° C. The tests are repeated several times, optionally at different pressures, to obtain a more accurate permeability value. Unless otherwise mentioned, the permeability remains constant whatever the ΔP (i.e. the pressure difference across the membrane).

### Example 1: Hydrogen/1,1,1,2,3,3-Hexafluoropropane Separation

The experimental protocol detailed above was employed independently for each compound of the mixture under consideration: hydrogen (H2) and 1,1,1,2,3,3-hexafluoropropane (HFC-236ea). The membrane used is made of polypropylene, polymethylpentene, poly(phenylene oxide), polyimide. The results are shown in table 2 below. The permeability value is expressed in barrers. The selectivity mentioned in the table corresponds to the ratio of the permeabilities measured for the two species under consideration.

TABLE 2

| | Permeability (barrer) | | Selectivity |
|---|---|---|---|
| | H2 | HFC-236ea | H2/HFC-236ea |
| PP | 5.7 | 0.1 | 57 |
| PMP | 130 | 0.1 | 1300 |
| Polyimide | 2.8 | 0.5 | 5.6 |
| PPO | 118833 | 136 | 874 |

PP = polypropylene;
PMP = polymethylpentene;
PPO = poly(phenylene oxide)

As is shown by the data above, the membranes made of polyolefins or polyether are more permeable to hydrogen than to 1,1,1,2,3,3-hexafluoropropane. The membranes of polyolefin (polypropylene, polyethylene or polymethylpentene) or polyether type therefore make it possible to effectively separate hydrogen from HFC-236ea.

### Example 2: Nitrogen or Oxygen/HFC-236ea Separation

The experimental protocol detailed above was employed independently for each compound of the mixture under consideration: nitrogen (N2), oxygen (O2), 1,1,1,2,3,3-hexafluoropropane (HFC-236ea). The membrane used is made of polymethylpentene or polypropylene. The results are shown in table 3 below. The permeability value is expressed in barrers. The selectivity mentioned in the table corresponds to the ratio of the permeabilities measured for the two species under consideration.

TABLE 3

| | Permeability (barrer) | | | Selectivity | |
|---|---|---|---|---|---|
| | N2 | O2 | HFC-236ea | N2/HFC-236ea | O2/HFC-236ea |
| PP | 3 | 13 | 0.1 | 30 | 130 |
| PMP | 9 | 32 | 0.1 | 90 | 320 |

The results above show that the membranes made of polyolefin are permeable to nitrogen and oxygen rather than to 1,1,1,2,3,3-hexafluoropropane. Similar results were obtained with a membrane made of PPO. Thus, the membranes made of polyolefin and polyether make it possible to separate nitrogen and oxygen from 1,1,1,2,3,3-hexafluoropropane.

1. A process for purifying 1,1,1,2,3,3-hexafluoropropane from a mixture comprising 1,1,1,2,3,3-hexafluoropropane and hydrogen, said process comprising a step (a) of bringing said mixture into contact with a membrane M1 in order to form a flow F1 comprising said 1,1,1,2,3,3-hexafluoropropane and a flow F2 comprising hydrogen, characterized in that said membrane M1 is made of a material selected from the group consisting of polyolefin, polyether, polyimide, polyvinylidene fluoride, a cellulose-based material and poly(methyl methacrylate).

2. The process as claimed in claim 1, wherein said membrane M1 is made of a material selected from the group consisting of polyolefin and polyether.

3. The process as claimed in claim 2, wherein said membrane M1 is made of a material selected from the group consisting of polyethylene, polypropylene, polymethylpentene, poly[oxy(2,6-dimethyl-1,4-phenylene)] and poly(phenylene oxide).

4. The process as claimed in claim 3, wherein said membrane M1 has a selectivity of greater than 100, said selectivity being calculated by the ratio between the permeability of hydrogen and the permeability of said 1,1,1,2,3,3-hexafluoropropane through said membrane M1.

5. The process as claimed in claim 1, wherein said mixture contains less than 100 ppm of water based on the total weight of said mixture.

6. The process as claimed in claim 1, wherein the content by mass of hydrogen in said mixture is less than 25% by weight based on the total weight of said mixture.

7. The process as claimed in claim 1, wherein said mixture is obtained from a reaction of hydrogenation of hexafluoropropene, which has optionally been purified beforehand.

8. A process for producing 1,1,1,2,3,3-hexafluoropropane, comprising the steps of:
A) gas-phase hydrogenation of hexafluoropropene in the presence of hydrogen and of a hydrogenation catalyst in order to form a stream A1 comprising 1,1,1,2,3,3-hexafluoropropane and unreacted hydrogen;
B) optionally purification of the stream A1 in order to form a purified stream A2;
C) implementation of the purification process as claimed in claim 1 using said stream A1 or said stream A2.

9. A process for purifying 1,1,1,2,3,3-hexafluoropropane from a mixture comprising 1,1,1,2,3,3-hexafluoropropane and at least one contaminant selected from the group consisting of nitrogen and oxygen or a mixture thereof, said process comprising a step (a) of bringing said mixture into contact with a membrane M1′ in order to form a flow F1′ comprising said 1,1,1,2,3,3-hexafluoropropane and a flow F2′ comprising said at least one contaminant.

10. The process as claimed in claim 9, wherein said membrane M1′ is made of a material selected from the group consisting of polyolefin, polyether, polyimide, polyaramid, polyamide, polysulfone, polyvinylidene fluoride, poly(methyl methacrylate), polytetrafluoroethylene, polyvinyl fluoride, polychlorotrifluoroethylene, polyethylene-tetrafluoroethylene or tetrafluoroethylene/perfluorovinyl ether copolymer optionally substituted by an SO3H group, of a cellulose-based material and of a material containing a siloxane functional group.

11. The process as claimed in claim 10, wherein said contaminant is oxygen and said membrane M1′ is made of a material selected from the group consisting of polyolefin, polyether, polyimide, polyvinylidene fluoride, a cellulose-based material, polyalkylsiloxane and poly(methyl methacrylate).

12. The process as claimed in claim 11, wherein said membrane M1′ is made of a material selected from the group consisting of polyethylene, polypropylene, polymethylpentene, poly[oxy(2,6-dimethyl-1,4-phenylene)], poly(phenylene oxide), cellulose acetate, polyimide and polydimethylsiloxane.

13. The process as claimed in claim 11, wherein said contaminant is nitrogen and said membrane M1′ is made of a material selected from the group consisting of polyolefin and of polyether.

14. The process as claimed in claim 11, wherein the content by mass of said contaminant in said mixture is less than 5% based on the total weight of said mixture.

15. A process for purifying 1,1,1,2,3,3-hexafluoropropane from a mixture comprising 1,1,1,2,3,3-hexafluoropropane, nitrogen, hydrogen and optionally oxygen, said process comprising a step (a) of bringing said mixture into contact with a membrane M2 in order to form a flow F3 comprising said 1,1,1,2,3,3-hexafluoropropane and a flow F4 comprising nitrogen, hydrogen and optionally oxygen, said membrane M2 being made of a material selected from the group consisting of polyethylene, polypropylene, polymethylpentene, poly[oxy(2,6-dimethyl-1,4-phenylene)] and poly(phenylene oxide).
